(19) United States
(12) Patent Application Publication
(10) Pub. No.: US 2019/0355455 A1
(43) Pub. Date: Nov. 21, 2019

(54) DOCUMENT TRACKING PANEL APPARATUS

(71) Applicant: VVC Holding Corporation, Seattle, WA (US)

(72) Inventors: Amanda Ropa Mander, Seattle, WA (US); Megan Pearson, Seattle, WA (US); Cynthia Sowder, Seattle, WA (US); Diya Deb, Seattle, WA (US)

(21) Appl. No.: 16/413,592

(22) Filed: May 15, 2019

Related U.S. Application Data
(60) Provisional application No. 62/671,832, filed on May 15, 2018.

(51) Int. Cl. G16H 15/00; G06F 9/451; G16H 10/60

(52) U.S. Cl. CPC G16H 15/00; G16H 10/60; G06F 9/451

(57) ABSTRACT

Certain examples provide a new, improved, different graphical user interface to track documentation availability and status for one or more patient encounters. Certain examples provide improved patient documentation as well as processing to enable a computer to retrieve, organize, process, display, and facilitate interaction with patient information and patient encounter information. An example document tracking panel apparatus includes memory and a processor to: generate an interface for display including a primary workspace, the primary workspace displaying health content for a patient; trigger, based on selection of a first element in the primary workspace, generation of a secondary panel to expand from the primary workspace; incorporate the first element in the secondary panel; and generate an actionable output from the secondary panel including the first element.

### CROSS-REFERENCE TO RELATED APPLICATIONS

This patent claims priority to U.S. Provisional Application Ser. No. 62/671,832, entitled “DOCUMENT TRACKING PANEL APPARATUS” which was filed on May 15, 2018, and is hereby incorporated herein by reference in its entirety.

### FIELD OF THE DISCLOSURE

This disclosure relates generally to a user interface generation apparatus and associated methods and, more particularly, to apparatus and associated methods for document user interface generation to track and build documentation for an encounter.

### BACKGROUND

Most electronic medical record systems combine completion of documentation with workflows to care for a patient. This documentation is a “necessary evil” but can slow down the physician who wants to just treat the patient and move on. By combining documentation and other patient content in a static record, information can be missed, treatment can be delayed, errors can occur (e.g., in a diagnosis, treatment, billing, etc.), and associated computer systems run slower, less optimally, and with many excess tasks.

### BRIEF SUMMARY

Certain examples provide apparatus, systems, and methods to dynamically drive a patient encounter and associated documentation.

Certain examples provide a document tracking panel apparatus including memory including instructions for execution and at least one processor. The at least one processor is to: generate an interface for display including a primary workspace, the primary workspace displaying health content for a patient; trigger, based on selection of a first element in the primary workspace, generation of a secondary panel to expand from the primary workspace; incorporate the first element in the secondary panel; and generate an actionable output from the secondary panel including the first element.

Certain examples provide a computer-readable storage medium including instructions which, when executed, cause at least one processor to at least: generate an interface for display including a primary workspace, the primary workspace displaying health content for a patient; trigger, based on selection of a first element in the primary workspace, generation of a secondary panel to expand from the primary workspace; incorporate the first element in the secondary panel; and generate an actionable output from the secondary panel including the first element.

Certain examples provide a computer-implemented method to drive graphical user interface transformation for a patient encounter. The example method includes: generating, by executing an instruction using at least one processor, an interface for display including a primary workspace, the primary workspace displaying health content for a patient; triggering, based on selection of a first element in the primary workspace and by executing an instruction using the at least one processor, generation of a secondary panel to expand from the primary workspace; incorporating, by executing an instruction using the at least one processor, the first element in the secondary panel; and generating, by executing an instruction using the at least one processor, an actionable output from the secondary panel including the first element.

### BRIEF DESCRIPTION OF THE DRAWINGS

FIG. 1 illustrates an example interface system.

FIGS. 2A-2E illustrate example interface configurations including a primary workspace and one or more supplemental panels triggered by and displayed with respect to the primary workspace.

FIG. 3 illustrates an example data flow driving user interface display and manipulation including generation and update of the primary and secondary panel(s) of FIGS. 2A-2E.

FIGS. 4A-5 depict example interfaces illustrating specific implementations of the primary workspace and secondary panel(s) of FIGS. 2A-2E.

FIG. 6 illustrates example implementation of the processor and display of FIG. 1 to drive the interface(s) of FIGS. 1-5 for a patient encounter.

FIGS. 7-8 illustrate flow diagrams of instructions for example methods/process(es) to drive the example system and interface(s) of FIGS. 1-6.

FIG. 9 is a block diagram of an example processor platform capable of executing instructions to implement the example systems and methods of FIGS. 1-8.

FIGS. 10-12 illustrate example operating environments in which the example systems and methods of FIGS. 1-8 can be implemented and/or otherwise executed.

The features and technical aspects of the system and method disclosed herein will become apparent in the following Detailed Description set forth below when taken in conjunction with the drawings in which like reference numerals indicate identical or functionally similar elements.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

When introducing elements of various embodiments of the present disclosure, the articles “a,” “an,” “the,” and “said” are intended to mean that there are one or more of the elements. The terms “comprising,” “including,” and “having” are intended to be inclusive and mean that there may be additional elements other than the listed elements.

As used herein, the terms “system,” “unit,” “module,” “engine,” etc., may include a hardware and/or software system that operates to perform one or more functions. For example, a module, unit, or system may include a computer processor, controller, and/or other logic-based device that performs operations based on instructions stored on a tangible and non-transitory computer readable storage medium, such as a computer memory. Alternatively, a module, unit, engine, or system may include a hard-wired device that performs operations based on hard-wired logic of the device. Various modules, units, engines, and/or systems shown in the attached figures may represent the hardware that operates based on software or hardwired instructions, the software that directs hardware to perform the operations, or a combination thereof.

### I. Overview

Many electronic medical record (EMR) systems combine completion of documentation with workflows to care for a patient. This documentation is a “necessary evil” but can slow down the physician who wants to just treat the patient and move on. By separating documentation from a patient treatment workflow, certain examples can provide a technological platform to the physician which enables the physician to have more flexibility to complete documentation either within the visit or at another time (e.g., before the visit, after the visit, etc.).

In certain examples, in a patient visit, when a physician enters a patient encounter, a list of elements required for documentation of that patient encounter are displayed as a check list to enable the physician to track which documentation has been completed and which documentation remains. For example, when a physician begins a patient encounter, a secondary panel slides out from a primary display panel (e.g., to/from the right of the clinician's workflow/workspace application/interface, to/from the left side, to/from the top, to/from the bottom, from the center, etc.), and the secondary panel displays documentation associated with a particular visit type. As the physician examines and/or otherwise interacts with the patient, this secondary/documentation panel updates to show the physician what items he/she has done and what remains incomplete. Thus, the secondary panel serves as a documentation check list, for example. Alternatively or in addition, documentation can be separated in a pop up or on another page of the interface. This design is valuable at least in part because it displays in the workspace and is also navigable, which keeps the user focused on one area rather than having to navigate away.

Thus, certain examples provide a separation in the integration of documentation and clinical workflows so that physicians can focus on their patient care first and document later. Other EMRs and associated interfaces do not provide this flexibility.

Certain examples provide a new, improved, different graphical user interface to track documentation availability and status, as well as drive workflow, for one or more patient encounters. Certain examples provide improved patient documentation as well as processing to enable a computer to retrieve, organize, process, display, and facilitate interaction with patient information and patient encounter information in a way that was not previously possible.

As will be described further below, certain examples can integrate with and operate in a variety of healthcare environments and impact a variety of healthcare scenarios and data through sensing, decision support, workflow management, and control. The following section provides some context and example environment for the presently disclosed technology described further in the subsequent section below.

### II. Example Healthcare Document and Encounter Tracking Interface Apparatus and Methods

Certain examples provide new computing technology driving a dynamic patient encounter interface that monitors information displayed on and interaction with a primary workspace or interface. For example, information loaded in the primary workspace and selected, modified, and/or otherwise interacted with by a user, operating system, other application, etc., is propagated to a secondary interface that pops up over, pops out of, displays to the side of, etc., the primary workspace.

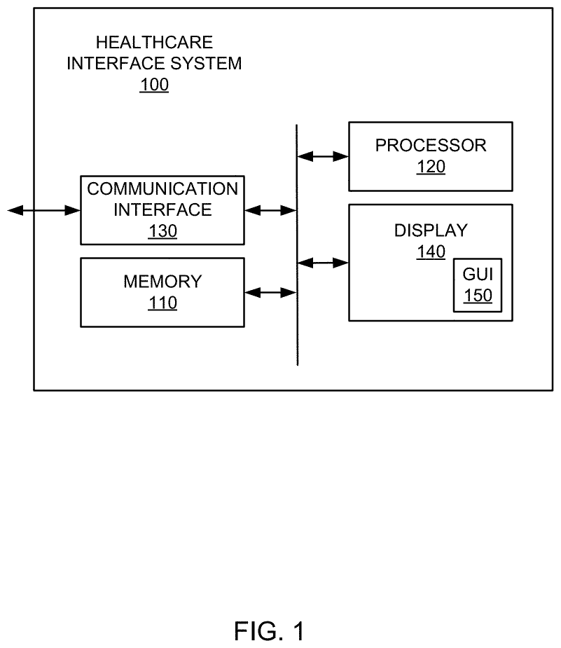

FIG. 1 illustrates an example interface system 100 including memory 110, a processor 120, a communication interface 130, and a display 140 with a graphical user interface 150. The example memory 110 stores program instructions for execution by the processor 120 to control the communication interface 130, process input and output of the communication interface 130, control the display 140, generate and update the graphical user interface 150, etc. The example system 100 can execute with and/or be implemented as part of another healthcare apparatus such as a physician desktop, radiology workstation, picture archiving and communication system, health information system, radiology information system, hospital information system, etc.

In certain examples, the processor 120 modifies the user interface 150 of the display 140 to provide a workspace for user interaction. For example, the workspace of the interface 150 can be generated by the processor 120 executing instruction(s) from memory 110 according to one or more data structures representing the workspace on the interface 150. The workspace can be configured with a patient imaging exam, laboratory test results, examination worklist, etc. As a user, application, other system, etc., interacts with the workspace of the interface 150, a secondary/auxiliary/supplemental panel can be triggered with respect to a primary panel of the workspace, for example. Rather than traditional graphical user interfaces, separately actionable by a user to access various functionality, the primary workspace and secondary panel(s) integrate a current application, user, and/or patient context to surface and propagate information, drive a workflow, and automatically generate a comprehensive record and/or checklist of a patient encounter, order, etc., for example.

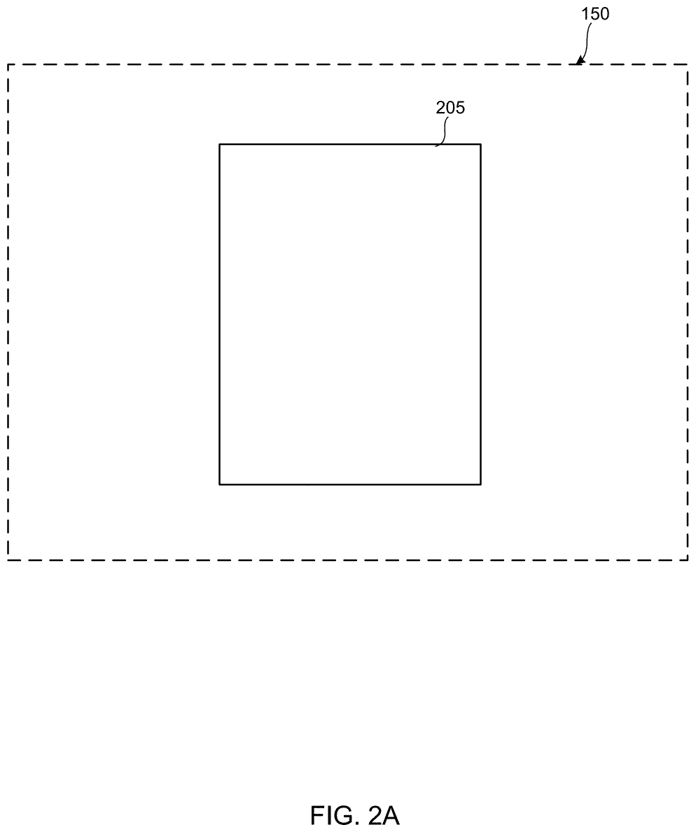

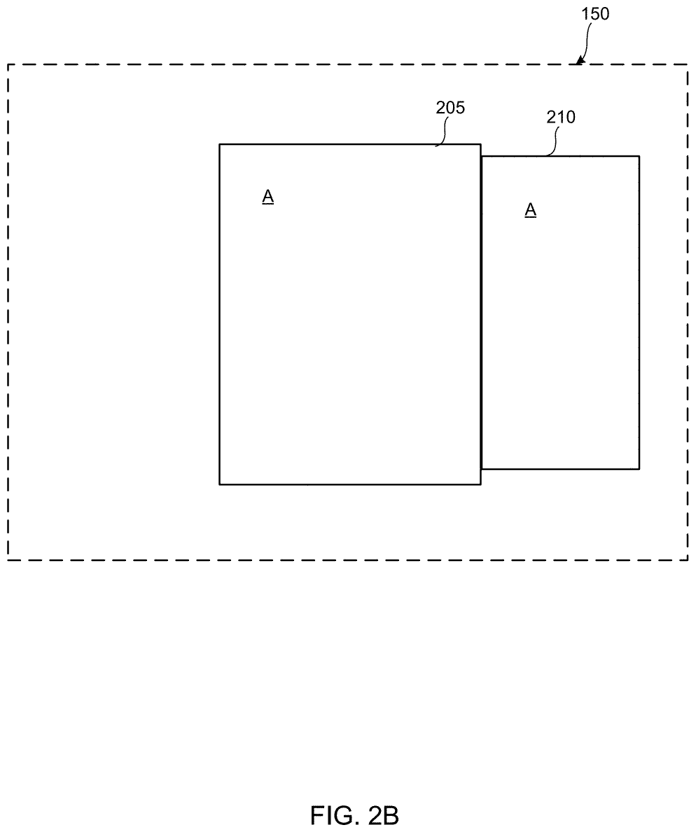

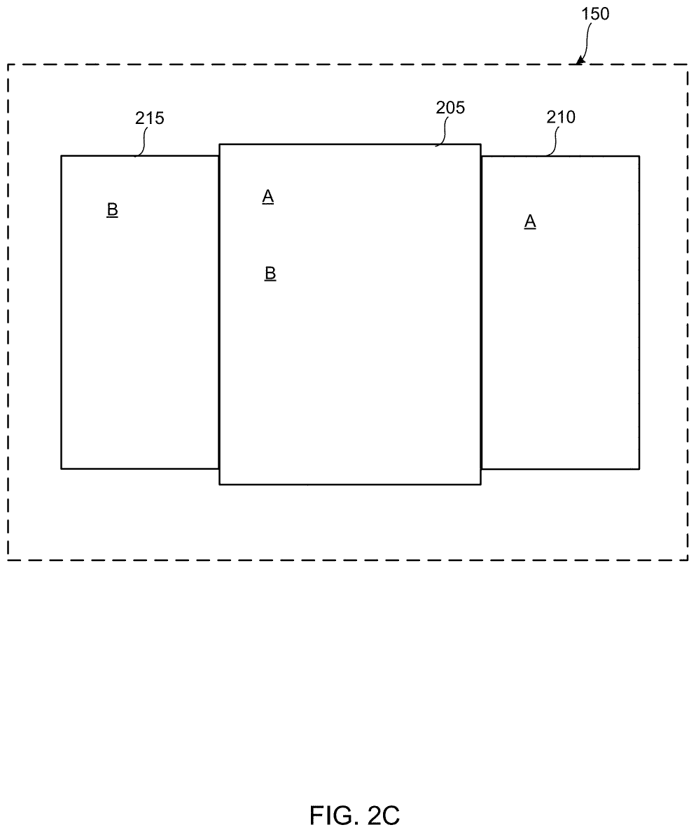

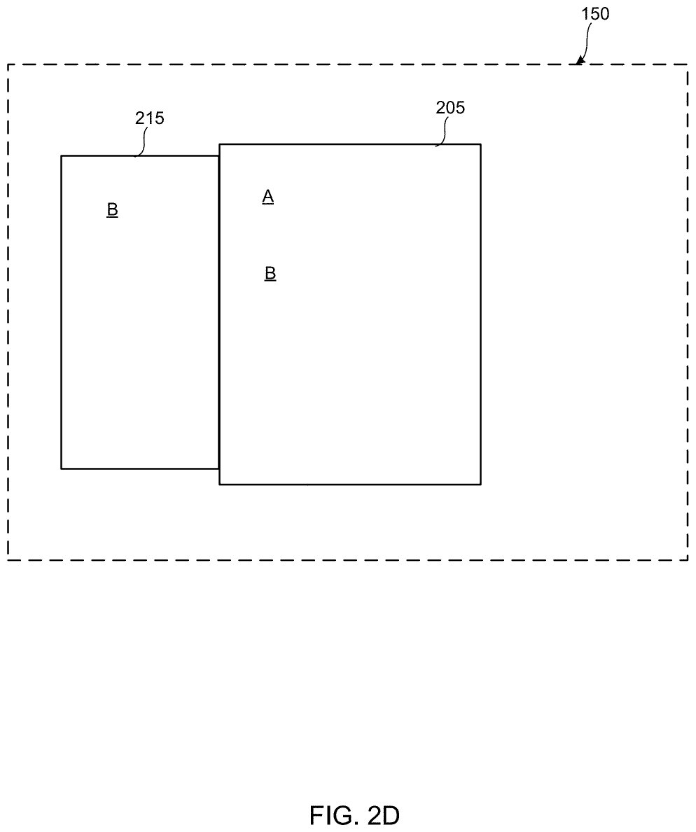

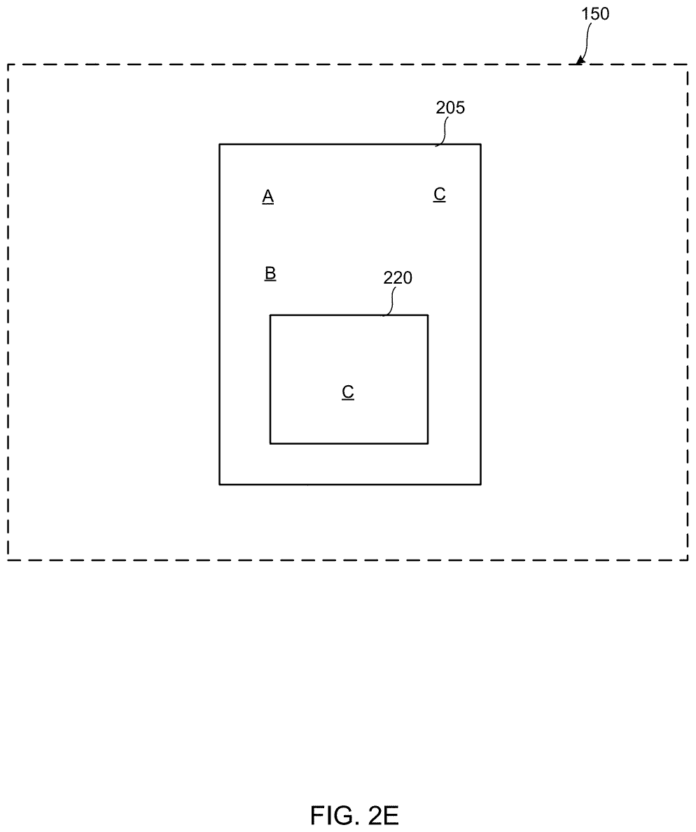

For example, FIGS. 2A-2E illustrate example configurations of the user interface 150 including a primary workspace 205 and one or more supplemental panels triggered by and displayed with respect to the primary workspace 205. FIG. 2A shows the primary workspace 205 displayed via the graphical user interface 150 on the example display 140. The primary workspace 205 can be (pre)loaded with content such as a clinician worklist, patient information (e.g., images, lab results, etc.), protocol/workflow template, etc.

As a user and/or other program interacts with the content and functionality of the workspace 205, the processor 120 triggers a secondary/supplemental panel 210 such as shown in the example of FIG. 2B. Interaction with content/functionality in the primary workspace 205 such as selecting a treatment option, confirming a lab result, recording a finding, etc., triggers the secondary panel 210 and a notation, indication, and/or entry, etc., in the secondary panel 210 regarding that interaction. For example, selecting a treatment option “A” in the primary workspace 205 triggers insertion and/or other notation of that treatment option “A” in the secondary panel 210 of the interface 150. As such, a record of selected options, indication of workflow/treatment, follow-up list of actions, trigger for additional application(s) and/or workflow action(s), etc., can be generated via the secondary panel 210.

In certain examples, such as FIG. 2C, multiple secondary panels 210, 215 can be generated to track different items “A” and “B” from the primary workspace 205. For example, the secondary panel 210 includes a recordation of selected treatment option(s) “A”, and the secondary panel 215 includes a subset of selected/highlighted patient electronic medical record results “B”. As shown in the example of FIG. 2D, a user and/or other application can close the secondary panel 210 such as manually and/or automatically when subject matter to which the secondary panel 210 relates has been completed. For example, when treatment option(s) have been completed in the primary workspace 205, the associated secondary panel 210 can close. Similarly, once the electronic medical record has been reviewed in the primary workspace 205, the associated secondary panel 215 can close. In certain examples, such as shown in FIG. 2E, a secondary panel 220 can pop-up over the primary panel 205, rather than or in addition to appearing on a side of the primary workspace panel 205.

Using the primary workspace 205 and secondary panel(s) 210-220, a process flow through a patient encounter can be streamlined with one primary screen 205 and supplemental pieces 210-220 that come in and out of the display 150 as needed while the workspace 205 remains. For example, information on medication, panel(s) 210-220 forming pieces of documentation for the patient encounter, etc., are provided to the user via the interface 150 and then go away while the primary workspace 205 remains.

Additionally, while some systems mix documentation and workflow, certain examples separate documentation into one or more secondary panels 210-220 from the primary workspace 205. It can be confusing, time-consuming, and/or error-prone to review, sign, and/or supplement documentation as a user clicks through forms and content in the primary workspace 205. Thus, in certain examples, the secondary panel(s) 210-220 separate from the primary workspace 205 and surface document(s) warranted for a particular visit/encounter type, context with other events/information, etc. The secondary panel(s) can track what has been done (e.g., a checklist, workflow execution, etc.) and capture evidence of completion into one or more supplemental panels for review and approval, for example. Thus, a user and/or other application does not need to rewrite information or instructions or track encounter progress manually because the system automatically captures it via the secondary panel(s) 210-220 and the processor 120, for example.

In certain examples, when an item (e.g., “A”, “B”, “C”, etc.) is selected in the secondary panel 210-220, an exam is automatically started, saving a large number of clicks/selections for clinicians and automatically placing the primary workspace 205 in a correct location, context, etc., to conduct a review, examination, other encounter, etc., and document an outcome. A user and/or application can begin an encounter with relevant documentation, and a workflow through the encounter can be linear and/or non-linear, with an order of screens and flow of information varying based on the patient, clinician, encounter, etc.

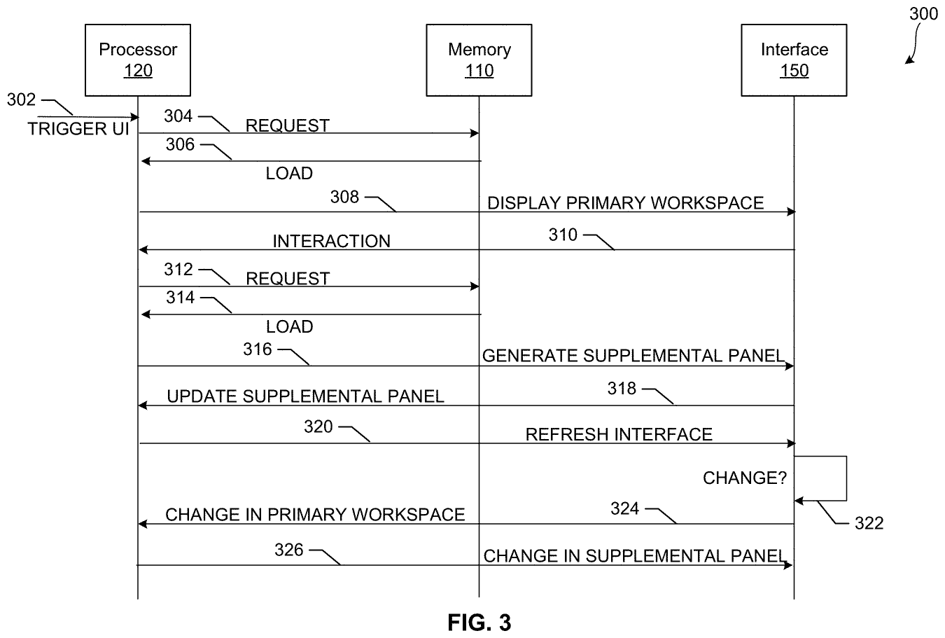

FIG. 3 illustrates an example data flow 300 driving user interface display and manipulation including generation and update of the primary 205 and secondary 210-220 panels. At 302, a trigger causes the processor 120 to begin interface 150 generation by requesting 304 information from memory 110. A load 306 from memory 110 enables the processor 120 to generate 308 a primary workspace 205 to be displayed via the user interface 150. The interface 150 is then available for interaction via the primary workspace 205, and interaction 310 is provided to the processor 120, which requests 312 corresponding information from memory 110. The requested content is loaded 314 for the processor 120, and the processor 120 uses the content to generate 316 the supplemental panel(s) 210-220 to be displayed via the user interface 150. The supplemental panel 210-220 is updated 318 based on an update and/or other interaction via the primary workspace 205 of the user interface 150. The processor 120 generates 320 an update or refresh of the interface 150 based on new information, interaction, application execution, etc. The state of the interface 150 is evaluated to detect a change 322 in the interface 150. When a change 322 occurs, the change in primary workspace 205, for example, is relayed 324 from the user interface 150 of the display 140 to the processor 120. The processor 120 determines a corresponding change in the supplemental panel 210-220 based on the change in the primary workspace 205, and generates 326 an updated supplemental panel 210-220 for the interface 150.

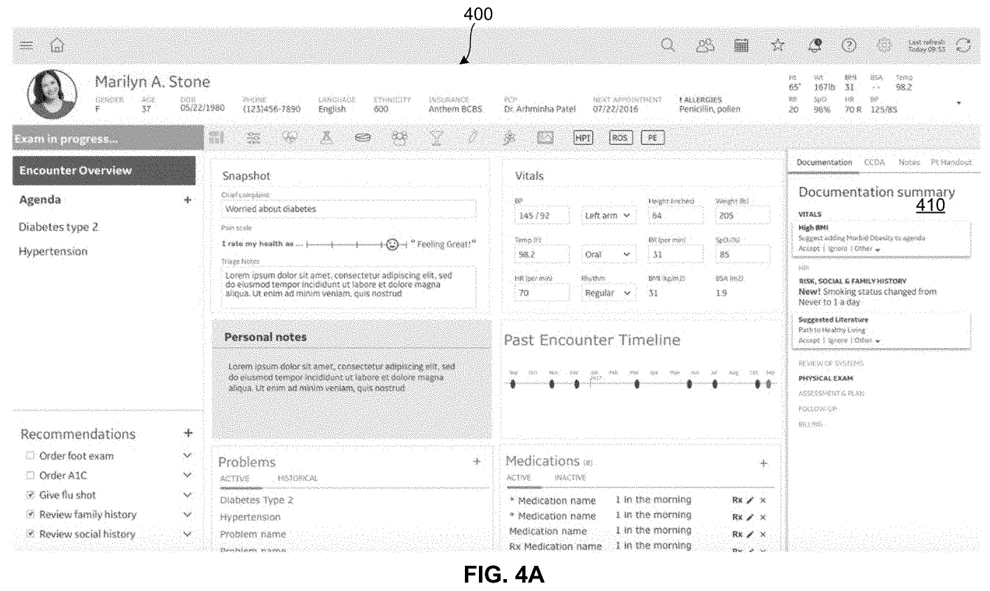

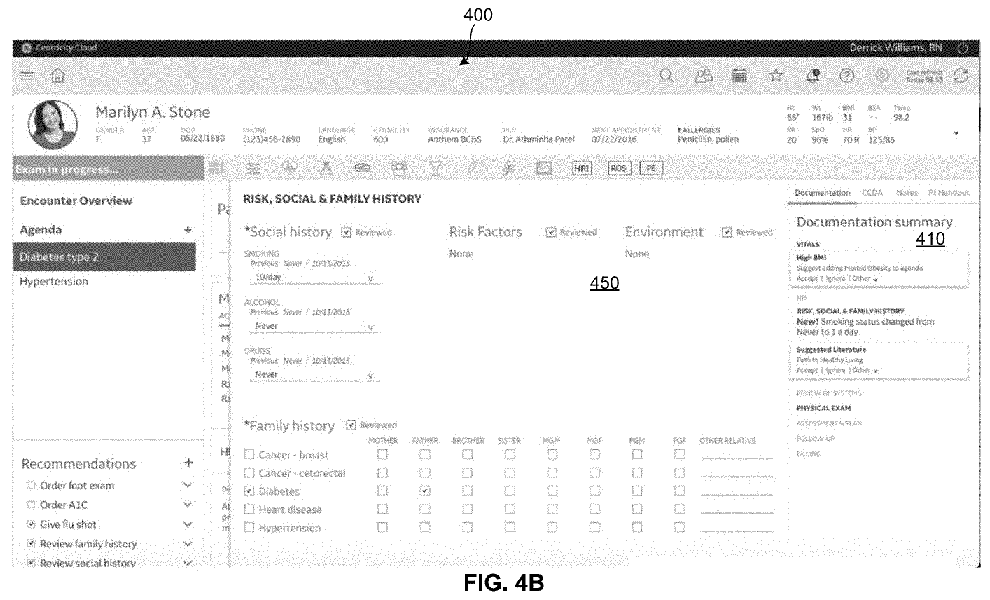

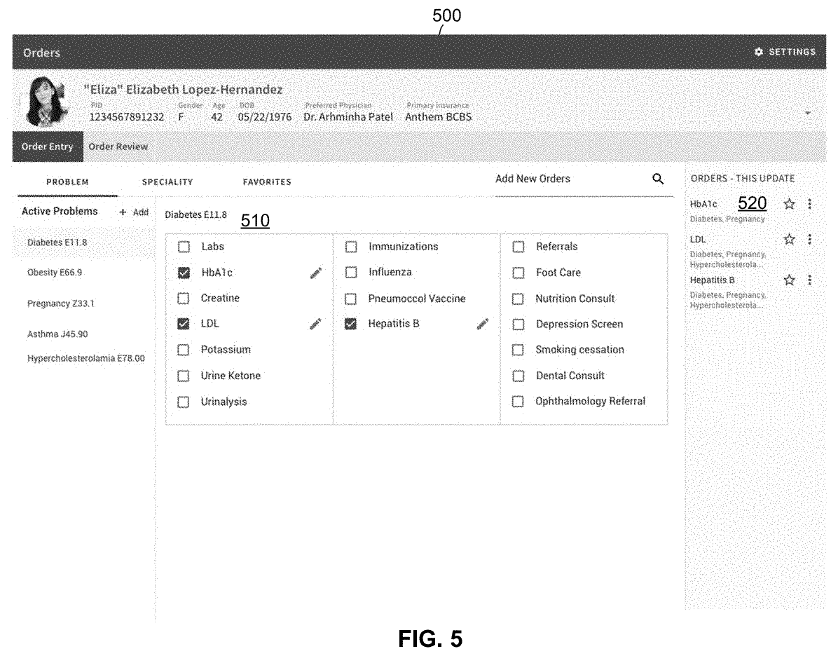

FIGS. 4A-5 depict example interfaces illustrating specific implementations of the primary workspace 205 and secondary panel(s) 210-220 described above with respect to FIGS. 2A-2E. FIG. 4A illustrates an example patient record interface 400 that opens a document summary interface portion 410 when an item on the interface 400 is clicked or otherwise selected. The documentation summary interface portion 410 provides a summary of available documents that have been determined to be relevant to one or more of the patient, healthcare practitioner, patient encounter, image/exam study, reason for exam, patient condition, patient care plan, healthcare protocol workflow, etc. For example, a heuristic and/or machine learning algorithm can be used to compare available documents to the one or more relevancy criteria (e.g., patient, healthcare practitioner, patient encounter, image/exam study, reason for exam, patient condition, patient care plan, healthcare protocol workflow, etc.) to generate a set of relevant documents to drive the summary interface portion 410. This document panel 410 forms a check list with respect to the actual documentation for the user, for example. The documentation panel 410 slides over and/or otherwise pops up to appear on or in the primary interface 400 so as to not distract the user's attention from the interface 400, 410 display (e.g., shown as part of the graphical user interface 150 on the display 140 in which the patient record interface 400 forms the primary workspace 205 and the documentation summary panel 410 is a secondary panel 210, etc.). The panel 410 may obscure content on the underlying interface 400 and/or may displace such content by compacting the rest of the interface 400, etc.

In certain examples, the documentation panel 410 can be populated based on items selected in the primary interface 400. Thus, if vitals information comes in and the physician selects it for review, it populates the documentation panel 410 so that the user remembers to look at the vitals later and has easy access to that documentation by selection through the panel 410, for example. Thus, if a user interacts with vitals information or vitals is updated (e.g., patient has a high body mass index (BMI), etc.), an indication of the vitals information and change in/alert associated with the vitals information (e.g., high BMI, etc.) is added to the documentation summary panel 410 to form an actionable record of the change in and/or other access to information, for example. If history of past illness (HPI) information comes in (e.g., through the examining clinician and/or other documentation), that documentation can be noted in the panel 410 for review, addition to the patient's EMR, etc. Other categories such as review of systems, physical exam, assessment and plan, follow-up, billing, etc., can be present in the panel 410 and dynamically generated for follow-up by the user, for example. In certain examples, the panel 410 can include consolidated clinical document architecture (CCDA) templates/guides, notes, patient handout information, etc., as well as the documentation summary.

In certain examples, further information can be viewed from the documentation summary 410 by selecting an entry, link, icon, representation, and/or other information from the summary 410 and/or from the primary interface 400. For example, FIG. 4B depicts an example social history panel 450 triggered from the summary panel 410 and displayed over the primary interface 400. The social history panel 450 provides further social history, family history, risk factors, environmental factors, etc., with respect to the patient. The social history panel 450 can be customized for relevance to the particular document in the documentation summary 410 and/or otherwise for the particular patient, healthcare practitioner, patient encounter, image/exam study, reason for exam, patient condition, patient care plan, healthcare protocol workflow, and/or other relevancy criterion, etc. The user can interact with the panel 450 to update, add, remove, and/or otherwise change content and options therein, for example. Changes to the social history panel 450 (e.g., change in smoking status, other risk factor, etc.) are noted in the documentation summary panel 410, for example.

Thus, certain examples provide apparatus, systems, computer-readable media, and associated methods to process, configure, and transform a graphical user interface and its underlying processing system to provide a dynamic document tracking and patient care interface with primary, secondary, tertiary, etc., levels of information and interaction within the bounds of the primary interface. Certain examples facilitate gathering of documentation and tasks relevant to a patient's care without distracting from patient care and interaction, thus prioritizing patient health, safety, and comfort while optimizing and/or otherwise enhancing ability for diagnosis and treatment. Certain examples provide varying levels of information, archiving, reminders, content gathering and completion, etc., for improved display and processing of patient encounters.

Certain examples drive a variety of patient workflows. For example, in one workflow, a provider receives a call from a patient who needs a refill of a medication. The patient is not on the provider's schedule, so the patient needs to be located. The patient's record can be located and selected to land on that patient's landing page (e.g., the primary interface 400, etc.). In an example, upcoming appointments are on the left, along with open communications and documents. A central area is a customizable space which the provider can configure as he or she wants to view available information. Actions, such as creating a chart update for the patient, can be automatically triggered based on opening of the patient's record, update of information, access to the documentation summary 410, etc. The provider can then take action on the chart, for example. A read-only view can convert into an editable document/area of the interface (e.g., social history panel 450, etc.), for example. Relevant documents for the action being taken can populate the summary panel 410, for example.

In another example workflow, a patient's chart is prepared prior to a patient visit. The provider visits the same patient landing patient where the record for that patient has been retrieved. The interface 400 provides updates as events occur (e.g., an urgent care visit, etc.), and the provider can view and interact with information related to those event(s), for example. For example, the provider can select to view a note from an external clinic, etc., that has been added to that patient's record. The provider can view follow-up items from that external event and can determine his or her own items for follow-up. When the provider selects an item on the interface 400 for follow-up, the agenda or summary 410 is automatically populated with that item and/or document(s) related to action on that item for the next patient encounter. Additionally, relevant fields for such item(s) can be automatically populated based on the patient, the provider, patient history, family history, protocol, reason for exam, etc. The provider can customize such information and sign and save for the record and future patient encounter, for example.

In another example workflow, during the patient encounter, the provider has the patient agenda and can select the patient to go straight to a patient encounter interface, bypassing the patient landing page. From the interface 400, the provider can view documents 410, take action on items/issues, etc. For example, the provider can select HPI in the summary panel 410 and add information regarding the patient's HPI. The provider can cycle through available sections using arrows, etc., and/or select a particular area to open, for example.

Thus, certain examples provide a variety of interface views including a generic patient view that is problem-agnostic and a problem-specific patient context view.

Certain examples provide an aggregate medication view listing a patient's medications and allowing the provider to adjust them. The provider can access the view and return back to the original workspace through selection or clicking, etc. Medication and/or other information can be viewed a part of a provider workflow to capture assessments, care plans, etc. Once signed, the record can be saved, action (e.g., prescription, scheduling, discharge, admittance, etc.) can be triggered.

FIG. 5 illustrates an example order interface 500 forming all or part of the user interface 150 of the display 140. As shown in the example of FIG. 5, the order interface 500 includes a diabetes order panel 510 including a plurality of options for selection to generate an order for laboratory tests to evaluate a patient's diabetic condition. As shown in the example of FIG. 5, selecting options in the order panel 510 triggers a supplemental panel 520 showing the subset of selected diabetes test options selected and/or otherwise activated in the primary order panel 510. In the example of FIG. 5 selection of HbAlc, LDL, and Hepatitis B in the primary order panel 510 triggers the processor 120 to generate secondary panel 520 reflecting the order to be send for lab tests. Thus, interaction with the primary order panel 510 generates an order in the secondary panel 520 which can be saved, edited, submitted for approval/fulfillment, etc.

Thus, certain examples provide an interface and associated panels enabling a streamlined flow through a patient encounter that did not previously exist. For example, a primary screen is provided for interaction, and supplemental pieces move in and out as needed while the user stays on the primary workspace screen. Thus, items (e.g., medication, relevant documentation) are brought to the user's attention for review/interaction, and then they go away and the user is still in their workspace.

While EMRs provide mixed documentation and workflow for a provider, the provider must review and sign every form available. Certain examples instead provide a documentation panel and surface only documents needed for a visit type and/or other context. Document and/or other task completion is tracked (e.g., like a checklist) and content is captured and converted into a review and sign sheet to be signed by the provider, who does not have to rewrite content because it has been captured and transformed into the document for user review/approval.

Certain examples enable triggering of an action by selection of an item from the primary workspace 400, 450, 510 and/or a panel 410, 520. For example, a click and/or other selection can automatically start an exam and put the provider in the appropriate, relevant context and workspace so that what they do is documented. For example, a selection can transform the interface 400 from a read-only view to starting a patient encounter with documentation to be manipulated. Linear and non-linear workflows can be supported, and screen order can vary.

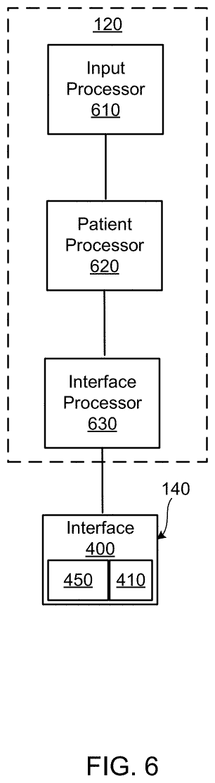

FIG. 6 illustrates example implementation of the processor 120 and display 140 to drive the interface(s) 150, 400-500 for a patient encounter. The example implementation of the processor 120 includes an input processor 610 to receive and organize (e.g., normalize, reformat, etc.) patient, visit, and/or other documentation input (e.g., from a PACS, RIS, EMR, archive, patient intake kiosk, etc.) from the memory 110 and/or communication interface 130 and provide the input to a patient processor 620, which processes the input patient data from the input processor 610 in conjunction with protocol information, provider preference, treatment guidelines, etc. to drive an interface processor 630. The interface processor 630 generates the interfaces 150, 400, 410, 450, 500, 510, 520 from the available patient information combined with template information to facilitate display of and interaction with patient content, provider content, reference material, diagnosis/treatment options, etc. Feedback and/or other action from the interface 150, 400, 500 (and its constituent panels 410, 450, 510, 520, etc.) is provided back to the patient processor 620 via the interface processor 630. In certain examples, the input processor 610 can provide an update back to its data source from the patient processor 620.

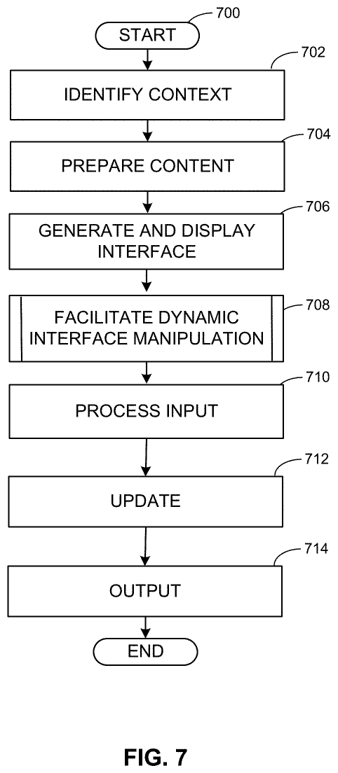

FIG. 7 illustrates a flow diagram of instructions for an example method or process 700 to drive the example system 100 and its interface(s) 150, 205-220, 400-500. At block 702, a context can be identified (e.g., unscheduled patient request, preparation for patient encounter, execution of patient encounter, etc.). At block 704, content can be prepared (e.g., patient medication/prescription information, patient record, external notes, reason for exam, protocol/treatment information, care plan, history, template, etc.) based on the identified context. At block 706, the interface 150, 400, 500 is generated and displayed from the prepared content. The context (e.g., radiology reading, patient encounter, lab order, exam order, etc.) can be used with the prepared content to generate the interface 400, for example.

At block 708, dynamic manipulation of the interface 150, 400, 500 is facilitated such as to spawn panel 205-220, 410, 450, 510, and/or 520, transform from read-only to editable/interaction, react to provider modification, etc. For example, the primary workspace 205 of the interface 150 can be dynamically manipulated to spawn secondary panel(s) 210-220, etc. In certain examples, interaction can transform the interface 150 from read-only to editable, directly through the primary workspace 205 and/or through the secondary panel(s) 210-220. Interaction can create linear workflows through a sequential series of interfaces and/or non-linear workflows in which some interface(s) are skipped or taken out of order given the information presented and interaction with panel(s) 205-22, 410, 450, 510, 520, etc.

At block 710, input from the interface 400-500 is processed. For example, patient information update, patient order, prescription, diagnosis, treatment/care plan, referral, reminder, order, approval, etc., provided through the interface 400-500 is processed and provided to another system (e.g., to trigger a next action such as prescription, admittance, discharge, referral, treatment, etc., for storage (e.g., in an EMR, enterprise archive, vendor neutral archive, PACS, RIS, specialty system, etc.), etc.). At block 712, content is updated (e.g., based on provider input/order, patient input, external input (e.g., clinic visit, image study, lab results, etc.), next action, new develop, interface modification, etc.). For example, input with respect to the primary workspace 205 can trigger an update of the secondary panel(s) 210-220, etc. At block 714, an actionable output is generated. The output can be formed from one or more secondary panels 210-220 displaying a record of information selected form the primary workspace 205, for example. The output can be an electronic medical record update, an order, discharge instructions, a trigger for another system/application, an executable workflow formed in the secondary panel 210-220 from a plurality of interface screens in the primary workspace 205, etc. The output is actionable because it is executable and/or can be used to trigger further execution of workflow, treatment, analysis, etc. based on the content of the output from the secondary panel(s) 210-220, for example.

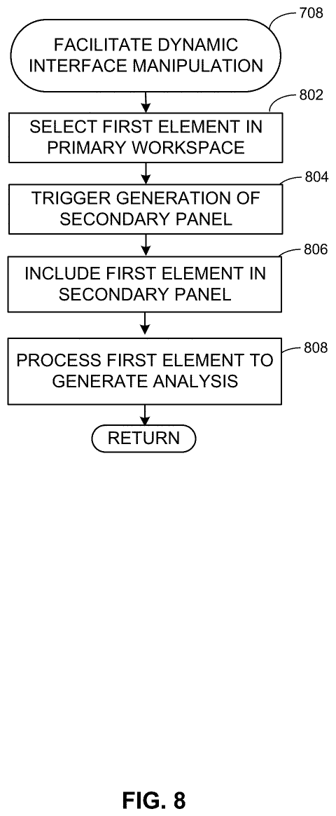

FIG. 8 illustrates an example implementation of facilitating dynamic manipulation of the interface 150 (e.g., block 708 of the example of FIG. 7). At block 802, a first element is selected in the primary workspace 205 of the interface 150. For example, a test, category of information, order, vital, image, etc., is selected from content displayed in the primary workspace 205. At block 804, generation of the secondary panel 210 is triggered by selection of the first element. For example, the secondary panel 210 pops out from the primary workspace 205 based on the selection of the first element. At block 806, the first element from the primary workspace 205 is included in the secondary panel 210. For example, the first element is displayed in the secondary panel 210 to document the first element selected from the primary workspace 205. In certain examples, a summarization or representation of the first element is included in the secondary panel 210, rather than an exact copy of the first element.

At block 808, the first element is processed to generate analysis regarding the first element. The analysis can be included in the secondary panel 210-220 as well. The analysis can be an alert, alarm, etc., of an emergent, abnormal, and/or escalating condition warranting further attention, follow-up, action, etc. For example, vitals selected from the primary workspace 205 can be processed by the processor 120 to determine that the patient's BMI is high, and the secondary panel 210 can be updated to display the selected vitals as well as the indication of high BMI. As another example, social history can be selected from the primary workspace 205 and processed by the processor 120 to determine that the patient's smoking status has changed from never to one a day. The secondary panel 210 can be updated to display the social history as well as the change in smoking status, for example.

Thus, certain examples create linear workflows through a sequential series of interfaces and/or non-linear workflows in which some interface(s) are skipped or taken out of order given the information presented and interaction with panel(s) 205-22, 410, 450, 510, 520, etc. In certain examples, interaction can transform the interface 150 from read-only to editable, directly through the primary workspace 205 and/or through the secondary panel(s) 210-220. Such dynamic interface transformation is not done in the human mind and is not able to be done manually by a human user. Instead, the technology driven the user interface 150 via the processor 120, instructions in memory 110, and the display 140 creates a new, useful application and ability in the user interface 150 to dynamically generate, interrelate, and accommodate a variety of workflows, patient data, user activity/preference, etc.

In certain examples, a model, machine learning construct, other artificial intelligence network model, etc., can be used to learn which conditions, content, etc., in the primary workspace 205 trigger creation of which secondary panel(s) 210-220 to proactively trigger secondary panel interaction based on primary workspace 205 content.

One skilled in the art will appreciate that embodiments of the invention may be interfaced to and controlled by a computer readable storage medium having stored thereon a computer program. The computer readable storage medium includes a plurality of components such as one or more of electronic components, hardware components, and/or computer software components. These components may include one or more computer readable storage media that generally stores instructions such as software, firmware and/or assembly language for performing one or more portions of one or more implementations or embodiments of a sequence. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or electronic memory. Other forms of non-transitory and/or tangible computer readable storage media not list may be employed with embodiments of the invention.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

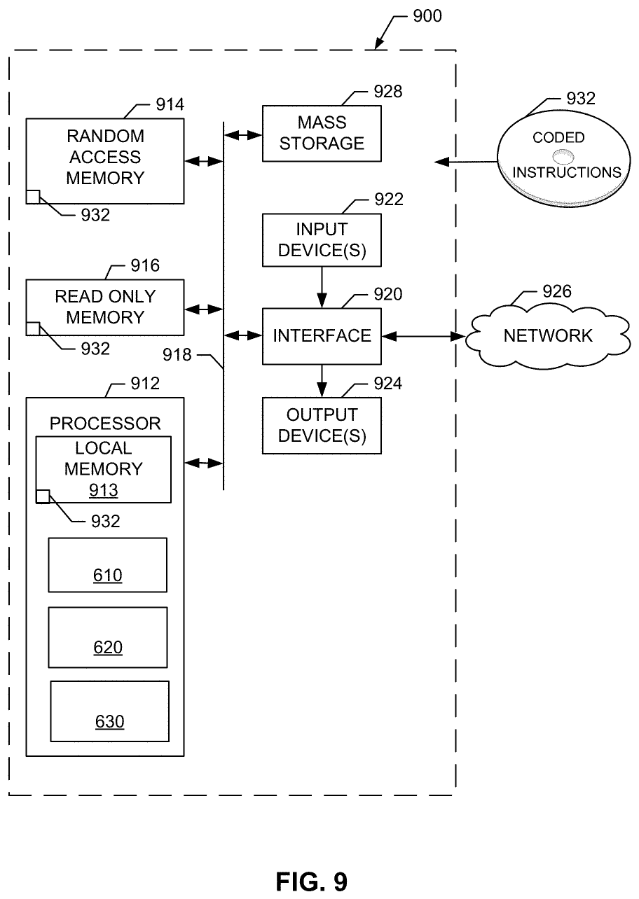

FIG. 9 is a block diagram of an example processor platform 900 capable of executing instructions to implement the example systems and methods of FIGS. 1-8. The processor platform 900 can be, for example, a server, a personal computer, a mobile device (e.g., a cell phone, a smart phone, a tablet such as an IPAD™), a personal digital assistant (PDA), an Internet appliance, or any other type of computing device.

The processor platform 900 of the illustrated example includes a processor 912. Processor 912 of the illustrated example is hardware. For example, processor 912 can be implemented by one or more integrated circuits, logic circuits, microprocessors or controllers from any desired family or manufacturer.

Processor 912 of the illustrated example includes a local memory 913 (e.g., a cache). Processor 912 of the illustrated example is in communication with a main memory including a volatile memory 914 and a non-volatile memory 916 via a bus 918. Volatile memory 914 can be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 916 can be implemented by flash memory and/or any other desired type of memory device. Access to main memory 914, 916 is controlled by a memory controller. The processor 912, alone or in conjunction with the memory 913, can be used to implement some or all of the example systems and associated methods disclosed herein.

Processor platform 900 of the illustrated example also includes an interface circuit 920. Interface circuit 920 can be implemented by any type of interface standard, such as an Ethernet interface, a universal serial bus (USB), and/or a PCI express interface.

In the illustrated example, one or more input devices 922 are connected to the interface circuit 920. Input device(s) 922 permit(s) a user to enter data and commands into processor 912. The input device(s) 922 can be implemented by, for example, an audio sensor, a microphone, a camera (still or video), a keyboard, a button, a mouse, a touchscreen, a track-pad, a trackball, isopoint and/or a voice recognition system.

One or more output devices 924 are also connected to interface circuit 920 of the illustrated example. Output devices 924 can be implemented, for example, by display devices (e.g., a light emitting diode (LED), an organic light emitting diode (OLED), a liquid crystal display, a cathode ray tube display (CRT), a touchscreen, a tactile output device, a light emitting diode (LED), a printer and/or speakers). Interface circuit 920 of the illustrated example, thus, typically includes a graphics driver card, a graphics driver chip or a graphics driver processor.

Interface circuit 920 of the illustrated example also includes a communication device such as a transmitter, a receiver, a transceiver, a modem and/or network interface card to facilitate exchange of data with external machines (e.g., computing devices of any kind) via a network 926 (e.g., an Ethernet connection, a digital subscriber line (DSL), a telephone line, coaxial cable, a cellular telephone system, etc.).

Processor platform 900 of the illustrated example also includes one or more mass storage devices 928 for storing software and/or data. Examples of such mass storage devices 928 include floppy disk drives, hard drive disks, compact disk drives, Blu-ray disk drives, RAID systems, and digital versatile disk (DVD) drives.

Coded instructions 932 associated with any of FIGS. 1-8 can be stored in mass storage device 928, in volatile memory 914, in the non-volatile memory 916, and/or on a removable tangible computer readable storage medium such as a CD or DVD.

### III. Example Operating Environments

Health information, also referred to as healthcare information and/or healthcare data, relates to information generated and/or used by a healthcare entity. Health information can be information associated with health of one or more patients, for example. Health information may include protected health information (PHI), as outlined in the Health Insurance Portability and Accountability Act (HIPAA), which is identifiable as associated with a particular patient and is protected from unauthorized disclosure. Health information can be organized as internal information and external information. Internal information includes patient encounter information (e.g., patient-specific data, aggregate data, comparative data, etc.) and general healthcare operations information, etc. External information includes comparative data, expert and/or knowledge-based data, etc. Information can have both a clinical (e.g., diagnosis, treatment, prevention, etc.) and administrative (e.g., scheduling, billing, management, etc.) purpose.

Institutions, such as healthcare institutions, having complex network support environments and sometimes chaotically driven process flows utilize secure handling and safeguarding of the flow of sensitive information (e.g., personal privacy). A need for secure handling and safeguarding of information increases as a demand for flexibility, volume, and speed of exchange of such information grows. For example, healthcare institutions provide enhanced control and safeguarding of the exchange and storage of sensitive patient PHI and employee information between diverse locations to improve hospital operational efficiency in an operational environment typically having a chaotic-driven demand by patients for hospital services. In certain examples, patient identifying information can be masked or even stripped from certain data depending upon where the data is stored and who has access to that data. In some examples, PHI that has been “de-identified” can be re-identified based on a key and/or other encoder/decoder.

A healthcare information technology infrastructure can be adapted to service multiple business interests while providing clinical information and services. Such an infrastructure may include a centralized capability including, for example, a data repository, reporting, discrete data exchange/connectivity, “smart” algorithms, personalization/consumer decision support, etc. This centralized capability provides information and functionality to a plurality of users including medical devices, electronic records, access portals, pay for performance (P4P), chronic disease models, and clinical health information exchange/regional health information organization (HIE/RHIO), and/or enterprise pharmaceutical studies, home health, for example.

Interconnection of multiple data sources helps enable an engagement of all relevant members of a patient's care team and helps improve an administrative and management burden on the patient for managing his or her care. Particularly, interconnecting the patient's electronic medical record and/or other medical data can help improve patient care and management of patient information. Furthermore, patient care compliance is facilitated by providing tools that automatically adapt to the specific and changing health conditions of the patient and provide comprehensive education and compliance tools to drive positive health outcomes.

In certain examples, healthcare information can be distributed among multiple applications using a variety of database and storage technologies and data formats. To provide a common interface and access to data residing across these applications, a connectivity framework (CF) can be provided which leverages common data and service models (CDM and CSM) and service oriented technologies, such as an enterprise service bus (ESB) to provide access to the data.

In certain examples, a variety of user interface frameworks and technologies can be used to build applications for health information systems including, but not limited to, MICROSOFT® ASP.NET, AJAX®, MICROSOFT® Windows Presentation Foundation, GOOGLE® Web Toolkit, MICROSOFT® Silverlight, ADOBE®, and others. Applications can be composed from libraries of information widgets to display multi-content and multi-media information, for example. In addition, the framework enables users to tailor layout of applications and interact with underlying data.

In certain examples, an advanced Service-Oriented Architecture (SOA) with a modern technology stack helps provide robust interoperability, reliability, and performance. Example SOA includes a three-fold interoperability strategy including a central repository (e.g., a central repository built from Health Level Seven (HL7) transactions), services for working in federated environments, and visual integration with third-party applications. Certain examples provide portable content enabling plug 'n play content exchange among healthcare organizations. A standardized vocabulary using common standards (e.g., LOINC, SNOMED CT, RxNorm, FDB, ICD-9, ICD-10, etc.) is used for interoperability, for example. Certain examples provide an intuitive user interface to help minimize end-user training. Certain examples facilitate user-initiated launching of third-party applications directly from a desktop interface to help provide a seamless workflow by sharing user, patient, and/or other contexts. Certain examples provide real-time (or at least substantially real time assuming some system delay) patient data from one or more information technology (IT) systems and facilitate comparison(s) against evidence-based best practices. Certain examples provide one or more dashboards for specific sets of patients. Dashboard(s) can be based on condition, role, and/or other criteria to indicate variation(s) from a desired practice, for example.

A. Example Healthcare Information System

An information system can be defined as an arrangement of information/data, processes, and information technology that interact to collect, process, store, and provide informational output to support delivery of healthcare to one or more patients. Information technology includes computer technology (e.g., hardware and software) along with data and telecommunications technology (e.g., data, image, and/or voice network, etc.).

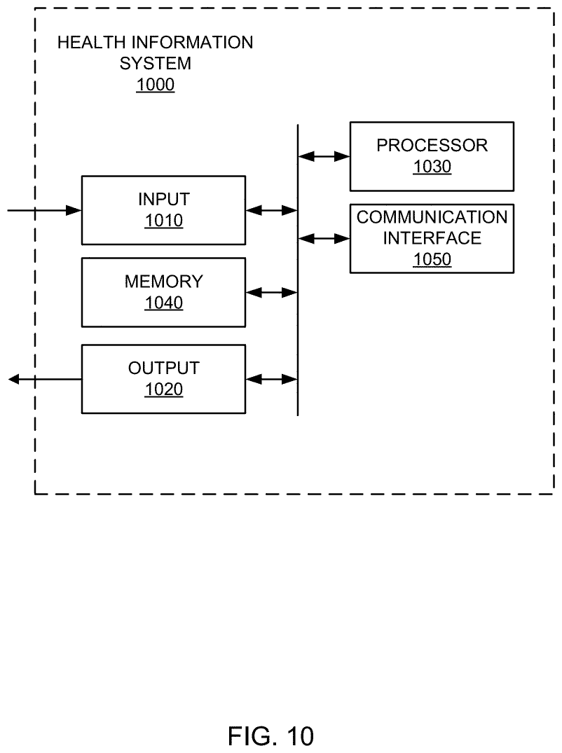

FIG. 10 shows a block diagram of an example healthcare-focused information system 1000. The example system 1000 can be used to implement the example health interface system 100, for example. The example system 1000 can be configured to implement a variety of systems and processes including image storage (e.g., picture archiving and communication system (PACS), etc.), image processing and/or analysis, radiology reporting and/or review (e.g., radiology information system (RIS), etc.), computerized provider order entry (CPOE) system, clinical decision support, patient monitoring, population health management (e.g., population health management system (PHMS), health information exchange (HIE), etc.), healthcare data analytics, cloud-based image sharing, electronic medical record (e.g., electronic medical record system (EMR), electronic health record system (EHR), electronic patient record (EPR), personal health record system (PHR), etc.), and/or other health information system (e.g., clinical information system (CIS), hospital information system (HIS), patient data management system (PDMS), laboratory information system (LIS), cardiovascular information system (CVIS), etc.

As illustrated in FIG. 10, the example information system 1000 includes an input 1010, an output 1020, a processor 1030, a memory 1040, and a communication interface 1050. The components of example system 1000 can be integrated in one device or distributed over two or more devices.

Example input 1010 may include a keyboard, a touch-screen, a mouse, a trackball, a track pad, optical barcode recognition, voice command, etc. or combination thereof used to communicate an instruction or data to system 1000. Example input 1010 may include an interface between systems, between user(s) and system 1000, etc.

Example output 1020 can provide a display generated by processor 1030 for visual illustration on a monitor or the like. The display can be in the form of a network interface or graphic user interface (GUI) to exchange data, instructions, or illustrations on a computing device via communication interface 1050, for example. Example output 1020 may include a monitor (e.g., liquid crystal display (LCD), plasma display, cathode ray tube (CRT), etc.), light emitting diodes (LEDs), a touch-screen, a printer, a speaker, or other conventional display device or combination thereof.

Example processor 1030 includes hardware and/or software configuring the hardware to execute one or more tasks and/or implement a particular system configuration. Example processor 1030 processes data received at input 1010 and generates a result that can be provided to one or more of output 1020, memory 1040, and communication interface 1050. For example, example processor 1030 can take user annotation provided via input 1010 with respect to an image displayed via output 1020 and can generate a report associated with the image based on the annotation. As another example, processor 1030 can process imaging protocol information obtained via input 1010 to provide an updated configuration for an imaging scanner via communication interface 1050.

Example memory 1040 may include a relational database, an object-oriented database, a data dictionary, a clinical data repository, a data warehouse, a data mart, a vendor neutral archive, an enterprise archive, etc. Example memory 1040 stores images, patient data, best practices, clinical knowledge, analytics, reports, etc. Example memory 1040 can store data and/or instructions for access by the processor 1030. In certain examples, memory 1040 can be accessible by an external system via the communication interface 1050.

Example communication interface 1050 facilitates transmission of electronic data within and/or among one or more systems. Communication via communication interface 1050 can be implemented using one or more protocols. In some examples, communication via communication interface 1050 occurs according to one or more standards (e.g., Digital Imaging and Communications in Medicine (DICOM), Health Level Seven (HL7), ANSI X12N, etc.). Example communication interface 1050 can be a wired interface (e.g., a data bus, a Universal Serial Bus (USB) connection, etc.) and/or a wireless interface (e.g., radio frequency, infrared (IR), near field communication (NFC), etc.). For example, communication interface 150 may communicate via wired local area network (LAN), wireless LAN, wide area network (WAN), etc. using any past, present, or future communication protocol (e.g., BLUETOOTH™, USB 2.0, USB 3.0, etc.).

In certain examples, a Web-based portal may be used to facilitate access to information, protocol library, imaging system configuration, patient care and/or practice management, etc. Information and/or functionality available via the Web-based portal may include one or more of order entry, laboratory test results review system, patient information, clinical decision support, medication management, scheduling, electronic mail and/or messaging, medical resources, etc. In certain examples, a browser-based interface can serve as a zero footprint, zero download, and/or other universal viewer for a client device.

In certain examples, the Web-based portal serves as a central interface to access information and applications, for example. Data may be viewed through the Web-based portal or viewer, for example. Additionally, data may be manipulated and propagated using the Web-based portal, for example. Data may be generated, modified, stored and/or used and then communicated to another application or system to be modified, stored and/or used, for example, via the Web-based portal, for example.

The Web-based portal may be accessible locally (e.g., in an office) and/or remotely (e.g., via the Internet and/or other private network or connection), for example. The Web-based portal may be configured to help or guide a user in accessing data and/or functions to facilitate patient care and practice management, for example. In certain examples, the Web-based portal may be configured according to certain rules, preferences and/or functions, for example. For example, a user may customize the Web portal according to particular desires, preferences and/or requirements.

B. Example Healthcare Infrastructure

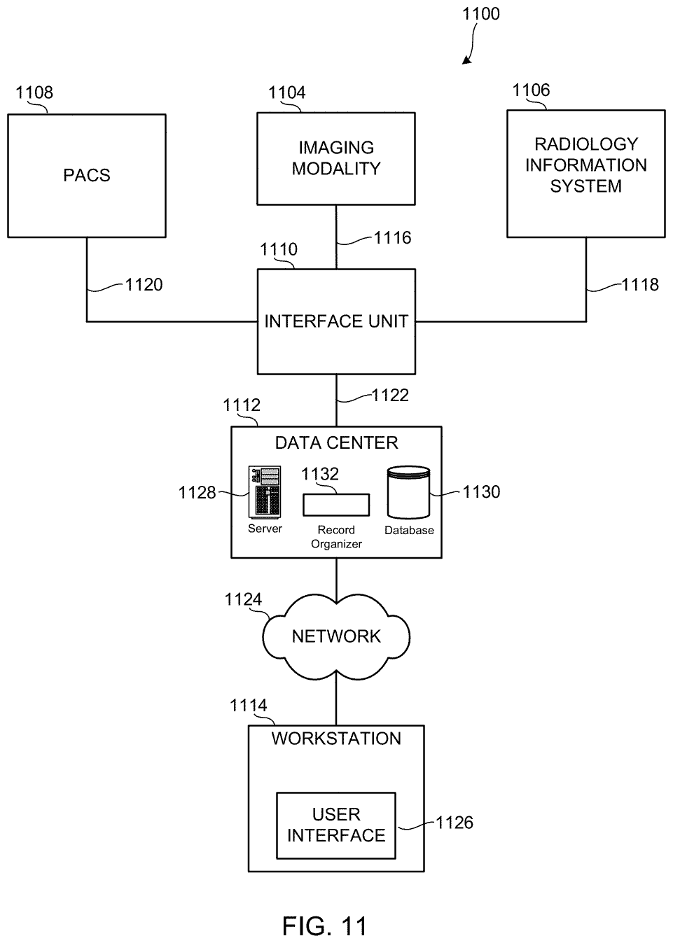

FIG. 11 shows a block diagram of an example healthcare information infrastructure 1100 including one or more subsystems such as the example healthcare-related information system 1000 illustrated in FIG. 10. Example healthcare system 1100 includes an imaging modality 1104, a RIS 1106, a PACS 1108, an interface unit 1110, a data center 1112, and a workstation 1114. In the illustrated example, scanner/modality 1104, RIS 1106, and PACS 1108 are housed in a healthcare facility and locally archived. However, in other implementations, imaging modality 1104, RIS 1106, and/or PACS 1108 may be housed within one or more other suitable locations. In certain implementations, one or more of PACS 1108, RIS 1106, modality 1104, etc., may be implemented remotely via a thin client and/or downloadable software solution. Furthermore, one or more components of the healthcare system 1100 can be combined and/or implemented together. For example, RIS 1106 and/or PACS 1108 can be integrated with the imaging scanner 1104; PACS 1108 can be integrated with RIS 1106; and/or the three example systems 1104, 1106, and/or 1108 can be integrated together. In other example implementations, healthcare system 1100 includes a subset of the illustrated systems 1104, 1106, and/or 1108. For example, healthcare system 1100 may include only one or two of the modality 1104, RIS 1106, and/or PACS 1108. Information (e.g., scheduling, test results, exam image data, observations, diagnosis, etc.) can be entered into the scanner 1104, RIS 1106, and/or PACS 1108 by healthcare practitioners (e.g., radiologists, physicians, and/or technicians) and/or administrators before and/or after patient examination. One or more of the imaging scanner 1104, RIS 1106, and/or PACS 1108 can communicate with equipment and system(s) in an operating room, patient room, etc., to track activity, correlate information, generate reports and/or next actions, and the like.

The RIS 1106 stores information such as, for example, radiology reports, radiology exam image data, messages, warnings, alerts, patient scheduling information, patient demographic data, patient tracking information, and/or physician and patient status monitors. Additionally, RIS 1106 enables exam order entry (e.g., ordering an x-ray of a patient) and image and film tracking (e.g., tracking identities of one or more people that have checked out a film). In some examples, information in RIS 1106 is formatted according to the HL-7 (Health Level Seven) clinical communication protocol. In certain examples, a medical exam distributor is located in RIS 1106 to facilitate distribution of radiology exams to a radiologist workload for review and management of the exam distribution by, for example, an administrator.

PACS 1108 stores medical images (e.g., x-rays, scans, three-dimensional renderings, etc.) as, for example, digital images in a database or registry. In some examples, the medical images are stored in PACS 1108 using the Digital Imaging and Communications in Medicine (DICOM) format. Images are stored in PACS 1108 by healthcare practitioners (e.g., imaging technicians, physicians, radiologists) after a medical imaging of a patient and/or are automatically transmitted from medical imaging devices to PACS 1108 for storage. In some examples, PACS 1108 can also include a display device and/or viewing workstation to enable a healthcare practitioner or provider to communicate with PACS 1108.

The interface unit 1110 includes a hospital information system interface connection 1116, a radiology information system interface connection 1118, a PACS interface connection 1120, and a data center interface connection 1122. Interface unit 1110 facilities communication among imaging modality 1104, RIS 1106, PACS 1108, and/or data center 1112. Interface connections 1116, 1118, 1120, and 1122 can be implemented by, for example, a Wide Area Network (WAN) such as a private network or the Internet. Accordingly, interface unit 1110 includes one or more communication components such as, for example, an Ethernet device, an asynchronous transfer mode (ATM) device, an 802.11 device, a DSL modem, a cable modem, a cellular modem, etc. In turn, the data center 1112 communicates with workstation 1114, via a network 1124, implemented at a plurality of locations (e.g., a hospital, clinic, doctor's office, other medical office, or terminal, etc.). Network 1124 is implemented by, for example, the Internet, an intranet, a private network, a wired or wireless Local Area Network, and/or a wired or wireless Wide Area Network. In some examples, interface unit 1110 also includes a broker (e.g., a Mitra Imaging's PACS Broker) to allow medical information and medical images to be transmitted together and stored together.

Interface unit 1110 receives images, medical reports, administrative information, exam workload distribution information, and/or other clinical information from the information systems 1104, 1106, 1108 via the interface connections 1116, 1118, 1120. If necessary (e.g., when different formats of the received information are incompatible), interface unit 1110 translates or reformats (e.g., into Structured Query Language (“SQL”) or standard text) the medical information, such as medical reports, to be properly stored at data center 1112. The reformatted medical information can be transmitted using a transmission protocol to enable different medical information to share common identification elements, such as a patient name or social security number. Next, interface unit 1110 transmits the medical information to data center 1112 via data center interface connection 1122. Finally, medical information is stored in data center 1112 in, for example, the DICOM format, which enables medical images and corresponding medical information to be transmitted and stored together.

The medical information is later viewable and easily retrievable at workstation 1114 (e.g., by their common identification element, such as a patient name or record number). Workstation 1114 can be any equipment (e.g., a personal computer) capable of executing software that permits electronic data (e.g., medical reports) and/or electronic medical images (e.g., x-rays, ultrasounds, MRI scans, etc.) to be acquired, stored, or transmitted for viewing and operation. Workstation 1114 receives commands and/or other input from a user via, for example, a keyboard, mouse, track ball, microphone, etc. Workstation 1114 is capable of implementing a user interface 1126 to enable a healthcare practitioner and/or administrator to interact with healthcare system 1100. For example, in response to a request from a physician, user interface 1126 presents a patient medical history. In other examples, a radiologist is able to retrieve and manage a workload of exams distributed for review to the radiologist via user interface 1126. In further examples, an administrator reviews radiologist workloads, exam allocation, and/or operational statistics associated with the distribution of exams via user interface 1126. In some examples, the administrator adjusts one or more settings or outcomes via user interface 1126.

Example data center 1112 of FIG. 11 is an archive to store information such as images, data, medical reports, and/or, more generally, patient medical records. In addition, data center 1112 can also serve as a central conduit to information located at other sources such as, for example, local archives, hospital information systems/radiology information systems (e.g., HIS 1104 and/or RIS 1106), or medical imaging/storage systems (e.g., PACS 1108 and/or connected imaging modalities). That is, the data center 1112 can store links or indicators (e.g., identification numbers, patient names, or record numbers) to information. In the illustrated example, data center 1112 is managed by an application server provider (ASP) and is located in a centralized location that can be accessed by a plurality of systems and facilities (e.g., hospitals, clinics, doctor's offices, other medical offices, and/or terminals). In some examples, data center 1112 can be spatially distant from the imaging modality 1104, RIS 1106, and/or PACS 1108. In certain examples, the data center 1112 can be located in the cloud (e.g., on a cloud-based server, an edge device, etc.).

Example data center 1112 of FIG. 11 includes a server 1128, a database 1130, and a record organizer 1132. Server 1128 receives, processes, and conveys information to and from the components of healthcare system 1100. Database 1130 stores the medical information described herein and provides access thereto. Example record organizer 1132 of FIG. 11 manages patient medical histories, for example. Record organizer 1132 can also assist in procedure scheduling, for example.

Certain examples can be implemented as cloud-based clinical information systems and associated methods of use. An example cloud-based clinical information system enables healthcare entities (e.g., patients, clinicians, sites, groups, communities, and/or other entities) to share information via web-based applications, cloud storage and cloud services. For example, the cloud-based clinical information system may enable a first clinician to securely upload information into the cloud-based clinical information system to allow a second clinician to view and/or download the information via a web application. Thus, for example, the first clinician may upload an x-ray imaging protocol into the cloud-based clinical information system, and the second clinician may view and download the x-ray imaging protocol via a web browser and/or download the x-ray imaging protocol onto a local information system employed by the second clinician.

In certain examples, users (e.g., a patient and/or care provider) can access functionality provided by system 1100 via a software-as-a-service (SaaS) implementation over a cloud or other computer network, for example. In certain examples, all or part of system 1100 can also be provided via platform as a service (PaaS), infrastructure as a service (IaaS), etc. For example, system 1100 can be implemented as a cloud-delivered Mobile Computing Integration Platform as a Service. A set of consumer-facing Web-based, mobile, and/or other applications enable users to interact with the PaaS, for example.

C. Industrial Internet Examples

The Internet of things (also referred to as the “Industrial Internet”) relates to an interconnection between a device that can use an Internet connection to talk with other devices on the network. Using the connection, devices can communicate to trigger events/actions (e.g., changing temperature, turning on/off, providing a status, etc.). In certain examples, machines can be merged with “big data” to improve efficiency and operations, provide improved data mining, facilitate better operation, etc.

Big data can refer to a collection of data so large and complex that it becomes difficult to process using traditional data processing tools/methods. Challenges associated with a large data set include data capture, sorting, storage, search, transfer, analysis, and visualization. A trend toward larger data sets is due at least in part to additional information derivable from analysis of a single large set of data, rather than analysis of a plurality of separate, smaller data sets. By analyzing a single large data set, correlations can be found in the data, and data quality can be evaluated.

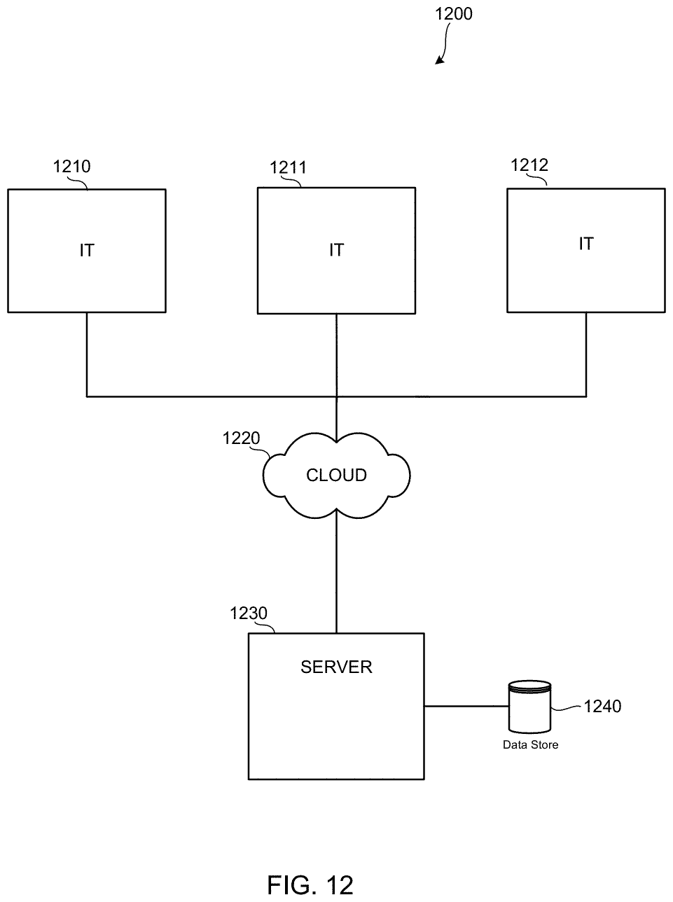

FIG. 12 illustrates an example industrial internet configuration 1200. Example configuration 1200 includes a plurality of health-focused systems 1210-1212, such as a plurality of health information systems 1000 (e.g., PACS, RIS, EMR, etc.) communicating via industrial internet infrastructure 1200. Example industrial internet 1200 includes a plurality of health-related information systems 1210-1212 communicating via a cloud 1220 with a server 1230 and associated data store 1240.

As shown in the example of FIG. 12, a plurality of devices (e.g., information systems, imaging modalities, etc.) 1210-1212 can access a cloud 1220, which connects the devices 1210-1212 with a server 1230 and associated data store 1240. Information systems, for example, include communication interfaces to exchange information with server 1230 and data store 1240 via the cloud 1220. Other devices, such as medical imaging scanners, patient monitors, etc., can be outfitted with sensors and communication interfaces to enable them to communicate with each other and with the server 1230 via the cloud 1220.

Thus, machines 1210-1212 within system 1200 become “intelligent” as a network with advanced sensors, controls, analytical based decision support and hosting software applications. Using such an infrastructure, advanced analytics can be provided to associated data. The analytics combines physics-based analytics, predictive algorithms, automation, and deep domain expertise. Via cloud 1220, devices 1210-1212 and associated people can be connected to support more intelligent design, operations, maintenance, and higher server quality and safety, for example.

Using the industrial internet infrastructure, for example, a proprietary machine data stream can be extracted from a device 1210. Machine-based algorithms and data analysis are applied to the extracted data. Data visualization can be remote, centralized, etc. Data is then shared with authorized users, and any gathered and/or gleaned intelligence is fed back into the machines 1210-1212.

While progress with industrial equipment automation has been made over the last several decades, and assets have become ‘smarter,’ the intelligence of any individual asset pales in comparison to intelligence that can be gained when multiple smart devices are connected together. Aggregating data collected from or about multiple assets can enable users to improve business processes, for example by improving effectiveness of asset maintenance or improving operational performance if appropriate industrial-specific data collection and modeling technology is developed and applied.

In an example, an industrial asset can be outfitted with one or more sensors configured to monitor respective ones of an asset's operations or conditions. Data from the one or more sensors can be recorded or transmitted to a cloud-based or other remote computing environment. By bringing such data into a cloud-based computing environment, new software applications informed by industrial process, tools and know-how can be constructed, and new physics-based analytics specific to an industrial environment can be created. Insights gained through analysis of such data can lead to enhanced asset designs, or to enhanced software algorithms for operating the same or similar asset at its edge, that is, at the extremes of its expected or available operating conditions.

Systems and methods to manage industrial assets can include or can be a portion of an Industrial Internet of Things (IIoT). In an example, an IIoT connects industrial assets, such as turbines, jet engines, and locomotives, to the Internet or cloud, or to each other in some meaningful way. The systems and methods described herein can include using a “cloud” or remote or distributed computing resource or service. The cloud can be used to receive, relay, transmit, store, analyze, or otherwise process information for or about one or more industrial assets. In an example, a cloud computing system includes at least one processor circuit, at least one database, and a plurality of users or assets that are in data communication with the cloud computing system. The cloud computing system can further include or can be coupled with one or more other processor circuits or modules configured to perform a specific task, such as to perform tasks related to asset maintenance, analytics, data storage, security, or some other function.

However, the integration of industrial assets with the remote computing resources to enable the IIoT often presents technical challenges separate and distinct from the specific industry and from computer networks, generally. A given industrial asset may need to be configured with novel interfaces and communication protocols to send and receive data to and from distributed computing resources. Given industrial assets may have strict requirements for cost, weight, security, performance, signal interference, and the like such that enabling such an interface is rarely as simple as combining the industrial asset with a general purpose computing device.

To address these problems and other problems resulting from the intersection of certain industrial fields and the IIoT, embodiments may enable improved interfaces, techniques, protocols, and algorithms for facilitating communication with and configuration of industrial assets via remote computing platforms and frameworks. Improvements in this regard may relate to both improvements that address particular challenges related to particular industrial assets (e.g., improved imaging systems, medical records systems, analytics systems, etc.) that address particular problems related to use of these industrial assets with these remote computing platforms and frameworks, and also improvements that address challenges related to operation of the platform itself to provide improved mechanisms for configuration, analytics, and remote management of industrial assets.

The Predix™ platform available from GE is a novel embodiment of such Asset Management Platform (AMP) technology enabled by state of the art cutting edge tools and cloud computing techniques that enable incorporation of a manufacturer's asset knowledge with a set of development tools and best practices that enables asset users to bridge gaps between software and operations to enhance capabilities, foster innovation, and ultimately provide economic value. Through the use of such a system, a manufacturer of industrial assets can be uniquely situated to leverage its understanding of industrial assets themselves, models of such assets, and industrial operations or applications of such assets, to create new value for industrial customers through asset insights.

D. Data Mining Examples

Imaging informatics includes determining how to tag and index a large amount of data acquired in diagnostic imaging in a logical, structured, and machine-readable format. By structuring data logically, information can be discovered and utilized by algorithms that represent clinical pathways and decision support systems. Data mining can be used to help ensure patient safety, reduce disparity in treatment, provide clinical decision support, etc. Mining both structured and unstructured data from radiology reports, as well as actual image pixel data, can be used to tag and index both imaging reports and the associated images themselves.

E. Example Methods of Use

Clinical workflows are typically defined to include one or more steps or actions to be taken in response to one or more events and/or according to a schedule. Events may include receiving a healthcare message associated with one or more aspects of a clinical record, opening a record(s) for new patient(s), receiving a transferred patient, reviewing and reporting on an image, executing orders for specific care, signing off on orders for a discharge, and/or any other instance and/or situation that requires or dictates responsive action or processing. The actions or steps of a clinical workflow may include placing an order for one or more clinical tests, scheduling a procedure, requesting certain information to supplement a received healthcare record, retrieving additional information associated with a patient, providing instructions to a patient and/or a healthcare practitioner associated with the treatment of the patient, radiology image reading, dispatching room cleaning and/or patient transport, and/or any other action useful in processing healthcare information or causing critical path care activities to progress. The defined clinical workflows may include manual actions or steps to be taken by, for example, an administrator or practitioner, electronic actions or steps to be taken by a system or device, and/or a combination of manual and electronic action(s) or step(s). While one entity of a healthcare enterprise may define a clinical workflow for a certain event in a first manner, a second entity of the healthcare enterprise may define a clinical workflow of that event in a second, different manner. In other words, different healthcare entities may treat or respond to the same event or circumstance in different fashions. Differences in workflow approaches may arise from varying preferences, capabilities, requirements or obligations, standards, protocols, etc. among the different healthcare entities.

In certain examples, a medical exam conducted on a patient can involve review by a healthcare practitioner, such as a radiologist, to obtain, for example, diagnostic information from the exam. In a hospital setting, medical exams can be ordered for a plurality of patients, all of which require review by an examining practitioner. Each exam has associated attributes, such as a modality, a part of the human body under exam, and/or an exam priority level related to a patient criticality level. Hospital administrators, in managing distribution of exams for review by practitioners, can consider the exam attributes as well as staff availability, staff credentials, and/or institutional factors such as service level agreements and/or overhead costs.

Additional workflows can be facilitated such as bill processing, revenue cycle mgmt., population health management, patient identity, consent management, etc.

The disclosed methods, apparatus, and articles of manufacture improve the operation and efficiency of using a processor and/or other computing device to dynamically generate a multi-paneled user interface to drive processor healthcare workflow execution and documentation generation. The disclosed methods, apparatus, and articles of manufacture are accordingly directed to one or more improvements in the functioning of a computer, a processor, display and interface technology, etc.

Descriptors “first,” “second,” “third,” etc. are used herein when identifying multiple elements or components which may be referred to separately. Unless otherwise specified or understood based on their context of use, such descriptors are not intended to impute any meaning of priority, physical order or arrangement in a list, or ordering in time but are merely used as labels for referring to multiple elements or components separately for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for ease of referencing multiple elements or components.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A document tracking panel apparatus comprising:
memory including instructions for execution; and
at least one processor to:
generate an interface for display including a primary workspace, the primary workspace displaying health content for a patient;
trigger, based on selection of a first element in the primary workspace, generation of a secondary panel to expand from the primary workspace;
incorporate the first element in the secondary panel; and
generate an actionable output from the secondary panel including the first element.

2. The apparatus of claim 1, wherein the at least one processor is to transform the interface from a read-only primary workspace into an editable secondary panel based on the selection of the first element.

3. The apparatus of claim 1, wherein the processor is to configure the primary workspace to document a patient encounter via the secondary panel.

4. The apparatus of claim 1, wherein the processor is to configure the primary workspace to generate an order for a patient via the secondary panel.

5. The apparatus of claim 1, wherein the secondary panel is to expand from the primary workspace by at least one of extending adjacent to the primary workspace or popping up on top of the primary workspace.

6. The apparatus of claim 1, wherein the at least one processor is to analyze the first element and incorporate an analysis of the first element into the secondary panel.

7. The apparatus of claim 6, wherein the analysis includes an alert.

8. The apparatus of claim 1, wherein the secondary panel is to enable a non-linear workflow through the interface through collection of a plurality of elements from the primary workspace.

9. A computer-readable storage medium comprising instructions which, when executed, cause at least one processor to at least:
generate an interface for display including a primary workspace, the primary workspace displaying health content for a patient;
trigger, based on selection of a first element in the primary workspace, generation of a secondary panel to expand from the primary workspace;
incorporate the first element in the secondary panel; and
generate an actionable output from the secondary panel including the first element.

10. The computer-readable storage medium of claim 9, wherein the instructions, when executed, cause the at least one processor to transform the interface from a read-only primary workspace into an editable secondary panel based on the selection of the first element.

11. The computer-readable storage medium of claim 9, wherein the instructions, when executed, cause the at least one processor to configure the primary workspace to document a patient encounter via the secondary panel.

12. The computer-readable storage medium of claim 9, wherein the instructions, when executed, cause the at least one processor to configure the primary workspace to generate an order for a patient via the secondary panel.

13. The computer-readable storage medium of claim 9, wherein the secondary panel is to expand from the primary workspace by at least one of extending adjacent to the primary workspace or popping up on top of the primary workspace.

14. The computer-readable storage medium of claim 9, wherein the instructions, when executed, cause the at least one processor to analyze the first element and incorporate an analysis of the first element into the secondary panel.

15. The computer-readable storage medium of claim 9, wherein the secondary panel is to enable a non-linear workflow through the interface through collection of a plurality of elements from the primary workspace.

16. A computer-implemented method to drive graphical user interface transformation for a patient encounter, the method comprising:
generating, by executing an instruction using at least one processor, an interface for display including a primary workspace, the primary workspace displaying health content for a patient;
triggering, based on selection of a first element in the primary workspace and by executing an instruction using the at least one processor, generation of a secondary panel to expand from the primary workspace;
incorporating, by executing an instruction using the at least one processor, the first element in the secondary panel; and
generating, by executing an instruction using the at least one processor, an actionable output from the secondary panel including the first element.

17. The method of claim 16, further including transforming the interface from a read-only primary workspace into an editable secondary panel based on the selection of the first element.

18. The method of claim 16, wherein the secondary panel expanding includes expanding from the primary workspace by at least one of extending adjacent to the primary workspace or popping up on top of the primary workspace.

19. The method of claim 16, further including analyzing the first element and incorporating an analysis of the first element into the secondary panel.

20. The method of claim 16, further including enabling a non-linear workflow through the interface in the secondary panel through collection of a plurality of elements from the primary workspace.
